(19) United States
(12) Patent Application Publication
(10) Pub. No.: US 2008/0294051 A1
(43) Pub. Date: Nov. 27, 2008

(54) ULTRASONIC OPERATING APPARATUS

(76) Inventors: Machiko Koshigoe, Hino-shi (JP); Hideto Yoshimine, Hachioji-shi (JP)

Correspondence Address: OSTROLENK FABER GERB & SOFFEN, 1180 AVENUE OF THE AMERICAS, NEW YORK, NY, 100368403, US

(21) Appl. No.: 11/753,872

(22) Filed: May 25, 2007

(51) Int. Cl. A61B 8/00; H01L 41/00

(52) U.S. Cl. 600/459; 310/334

(57) ABSTRACT

The ultrasonic operating apparatus includes an ultrasonic transducer that generates ultrasonic vibrations, and a probe that has a distal end portion and a proximal end portion, the proximal end portion being coupled to the ultrasonic transducer, and to which ultrasonic vibrations output from the ultrasonic transducer are transmitted, where the probe has a probe main body having a length of an integral multiple of about a half wavelength of the ultrasonic vibrations and an adjusting portion that changes a weight and a dimension of the probe main body to adjust a resonance frequency of the probe main body.

### BACKGROUND OF THE INVENTION

The present invention relates to an ultrasonic operating apparatus that utilizes ultrasonic waves to perform a procedure such as incision or excision of body tissue, or solidification.

In general, an ultrasonic operating apparatus that utilizes ultrasonic waves to perform a procedure such as incision or excision of body tissue, or solidification has been known. In a vibration system in the ultrasonic surgical system, variations in resonance frequency occur due to material for a probe, working of a probe, or assembling thereof, and the like. Since the resonance frequency of the ultrasonic operating apparatus is caused to match with dimensions of current parts, variations occur due to a working tolerance. In general, since the resonance frequency of the ultrasonic operating apparatus cannot be adjusted after the probe has been worked, it is difficult to improve yield.

As one example of the ultrasonic surgical instrument, for example, an apparatus described in International Publication No. 00/62688 Pamphlet (Patent Document 1), a method for changing a length of a screw at a connection portion with a probe to adjust a frequency of a transducer.

### BRIEF SUMMARY OF THE INVENTION

According to an aspect of the present invention, there is provided an ultrasonic operating apparatus comprising an ultrasonic transducer that generates ultrasonic vibrations and a probe that has a distal end and a proximal end, the proximal end being coupled to the ultrasonic transducer, and that transmits ultrasonic vibrations output from the ultrasonic transducer, wherein the probe has a probe main body that has a length of an integral multiple of about a half wavelength of the ultrasonic vibrations and an adjusting portion that changes a weight and a dimension of the probe main body to adjust a resonance frequency of the probe main body.

It is preferable that the probe main body has a first probe-constituting unit, a second probe-constituting unit, and an adjusting member that is interposed between the first probe-constituting unit and the second probe-constituting unit for adjusting the resonance frequency of the probe main body, the adjusting member being disposed at an antinode position of the ultrasonic vibrations, and the adjusting member is for changing the adjusting member to adjust the resonance frequency of the probe main body.

It is preferable that the adjusting member has a weight member interposed between the first probe-constituting unit and the second probe-constituting unit.

It is preferable that the weight member has a coupling portion comprising one of at least welding, screw joining, and fitting at a coupling section between the first probe-constituting unit and the second probe-constituting unit, where the fitting is means for pressure-fitting, into a small-diameter hole, a shaft body with a diameter larger than the diameter of the small-diameter hole.

It is preferable that the first and second probe-constituting units are made from metal material, and the weight member is made from the same metal material as that used in the probe-constituting units in the probe main body.

It is preferable that the metal material is one of at least titanium alloy, duralumin, and stainless steel.

It is preferable that the first and second probe-constituting units are made from metal material, and the weight member is made from metal material different from the metal material used in the probe-constituting units in the probe main body.

It is preferable that the weight member has a plurality of replacement members with different weights that are different in at least one of a dimension in a lengthwise direction along an axial central direction of the probe main body and a dimension in a diametrical direction thereof, and the adjusting portion selects and uses one of the plurality of replacement members, thereby adjusting the resonance frequency of the probe main body.

It is preferable that the weight member has a plurality of replacement members that have the same dimension in a lengthwise direction along an axial central direction of the probe main body and the same dimension in a diametrical direction thereof, and the adjusting portion selects the number of replacement members and uses the selected replacement members to change the length and the weight of the probe main body, thereby adjusting the resonance frequency.

It is preferable that the weight member has a plurality of replacement members made from different metal materials and the adjusting portion selects and uses one of the replacement members, thereby adjusting the resonance frequency of the probe main body.

It is preferable that the probe main body has hollow ducts provided in the first probe-constituting unit, the second probe-constituting unit, and the adjusting member, respectively, and the hollow duct of the first probe-constituting unit, the hollow duct of the second probe-constituting unit, and the hollow duct of the adjusting member are in communication with one another.

It is preferable that the probe main body has an adjusting hole for resonance frequency, the adjusting hole being disposed at an antinode position of the ultrasonic vibrations, and the adjusting portion has a joining member that can be joined to the adjusting hole, the joining member changing the weight of the probe main body to adjust the resonance frequency.

It is preferable that the joining member has a coupling portion comprising one of at least welding, screw joining, and fitting at a coupling section with the adjusting hole, where the fitting is means for pressure-fitting, into a small-diameter hole, a shaft body with a diameter larger than the diameter of the small-diameter hole.

It is preferable that the probe main body has a hollow conduit caused to communicate with the adjusting hole, and the joining member is coupled to the adjusting hole to close the adjusting hole.

It is preferable that the joining member has a coupling portion comprising one of at least welding, screw joining, and fitting at a coupling section with the adjusting hole, where the fitting is means for pressure-fitting, into a small-diameter hole, a shaft unit with a diameter larger than the diameter of the small-diameter hole.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1A is a perspective view showing a whole schematic configuration of an ultrasonic operating apparatus according to a first embodiment of the present invention;

FIG. 1B is a sectional view of the ultrasonic surgical instrument, taken along line 1B-1B in FIG. 1A;

FIG. 2 is a side view showing a probe of the ultrasonic operating apparatus according to the first embodiment;

FIG. 3 is a vertical sectional view of a main section showing a coupling section of a first probe-constituting unit, a second probe-constituting unit, and a weight member in the ultrasonic operating apparatus according to the first embodiment;

FIG. 4 is an explanatory diagram for explaining replacement members for the weight member of a probe main body in the ultrasonic operating apparatus according to the first embodiment;

FIG. 5 is a side view showing a first modification of the probe main body in the ultrasonic operating apparatus according to the first embodiment;

FIG. 6 is a vertical sectional view of a main section showing a second modification of the probe main body of the ultrasonic operating apparatus according to the first embodiment;

FIG. 7 is a vertical sectional view of a probe main body according to a second embodiment of the present invention;

FIG. 8 is a vertical sectional view of a main section showing a coupling section of a first probe-constituting unit, a second probe-constituting unit, and a resonance frequency adjusting member of the probe main body according to the second embodiment of the present invention;

FIG. 9 is a sectional view of the coupling section, taken along line IX-IX in FIG. 8;

FIG. 10 is a perspective view showing a probe main body of an ultrasonic operating apparatus according to a third embodiment of the present invention;

FIG. 11 is a vertical sectional view of the probe main body according to the third embodiment;

FIG. 12 is a perspective view showing a probe main body of an ultrasonic operating apparatus according to a fourth embodiment of the present invention;

FIG. 13 is a vertical sectional view of a main section showing a state that an adjusting member has been fixed in an adjusting hole of a probe main body of the ultrasonic operating apparatus according to the fourth embodiment; and

FIG. 14 is a sectional view of the main section, taken along line XIV-XIV in FIG. 13.

### DETAILED DESCRIPTION OF THE INVENTION

### First Embodiment

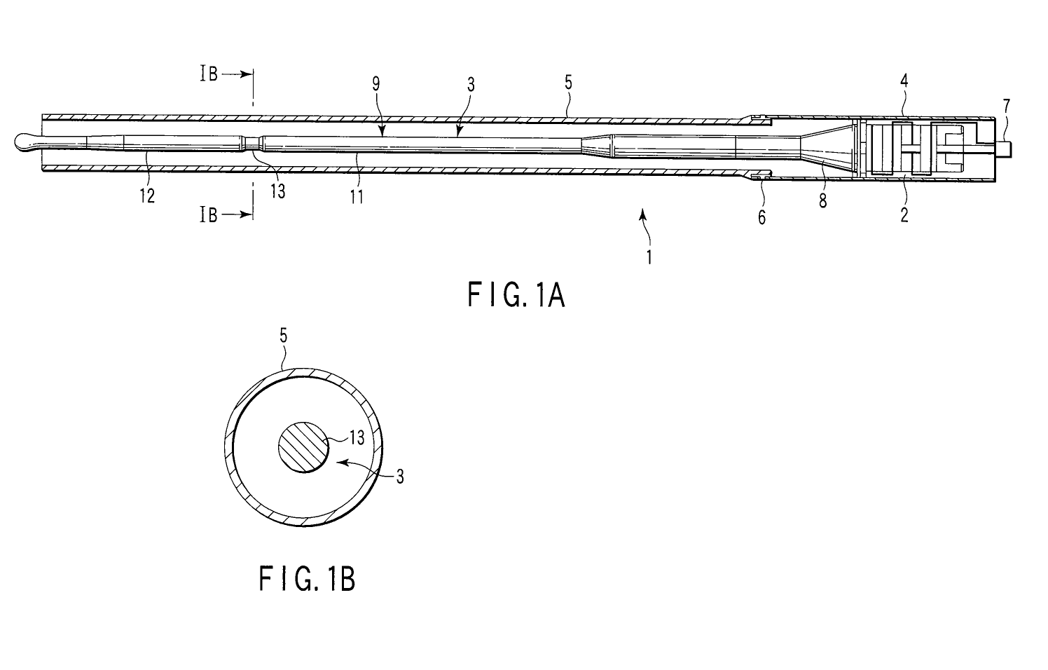

A first embodiment of the present invention will be explained below with reference to FIG. 1A to FIG. 4. FIG. 1A is a view showing a schematic configuration of a whole ultrasonic treatment tool 1 of an ultrasonic operating apparatus according to the present embodiment. The ultrasonic operating apparatus 1 has an ultrasonic transducer 2 that generates ultrasonic vibrations and a probe 3 that has a distal end portion and a proximal end portion, the proximal end portion being coupled to the ultrasonic transducer 2, and that transmits ultrasonic vibrations output from the ultrasonic transducer 2. An outer periphery of the ultrasonic transducer 2 is covered with a cylindrical transducer cover 4.

An outer periphery of the probe 3 is covered with a cylindrical sheath 5. As shown in FIG. 1B, the sheath 5 is disposed approximately coaxially with the probe 3. A distal end portion of the sheath 5 is coupled to a front end portion of the transducer cover 4 via a coupling ring 6. A terminal end portion of the ultrasonic transducer 2 is connected with one end portion of an electric cord 7. The other end portion of the electric cord 7 is connected to a power source main body (not shown).

A front end portion of the ultrasonic transducer 2 is coupled to a proximal end portion of a horn 8 that performs amplitude expansion of ultrasonic vibrations. The horn 8 is formed at its distal end portion with a screw hole (not shown) for probe attachment. A male screw (not shown) is formed on the proximal end portion of the probe 3. The male screw at the proximal end portion of the probe 3 is driven into a screw hole portion at a distal end portion of the horn 8.

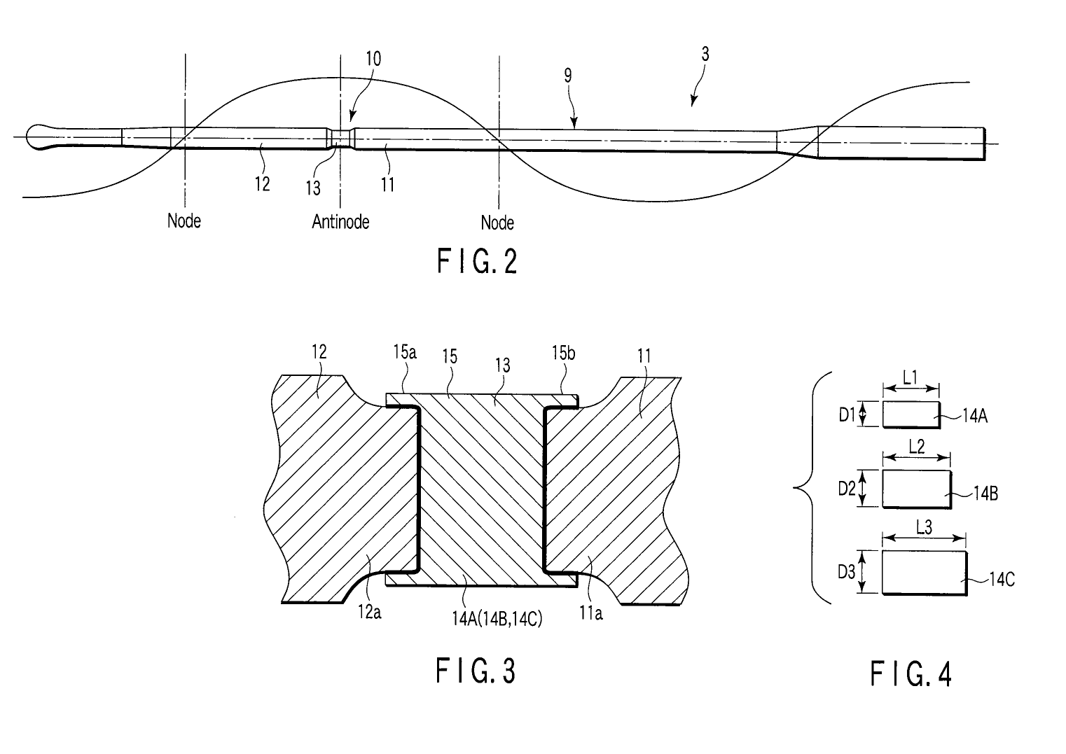

FIG. 2 is a sectional view showing an appearance of the whole probe 3. The probe 3 has a rod-like probe main body 9 that is an ultrasonic wave transmitting member with a length of an integral multiple of about a half wavelength of ultrasonic vibrations from the ultrasonic transducer 2 and an adjusting portion 10 that changes a weight and a dimension of the probe main body 9 to adjust a resonance frequency of the probe main body 9.

The probe main body 9 has a first probe-constituting unit 11, a second probe-constituting unit 12, and a weight member (adjusting member) 13 interposed between the first probe-constituting unit 11 and the second probe-constituting unit 12. The first and second probe-constituting units 11 and 12 are made from metal material such as titanium (Ti) alloy, duralumin, or stainless steel (SUS). The weight member 13 is made from at least one of the same metal material as material used in the first and second probe-constituting members 11 and 12 and metal material different in density from that in the first and second probe-constituting members 11 and 12. The weight member 13 is disposed at an antinode position of ultrasonic vibrations transmitted to the probe 3, as shown in FIG. 2.

The first probe-constituting unit 11 is disposed on the side of the proximal end portion of the probe main body 9 (on the side of a connecting portion with the ultrasonic transducer 2). The second probe-constituting unit 12 is a disposed at a front portion of the first probe-constituting unit 11.

As shown in FIG. 3, a front end portion of the first probe-constituting unit 11 is formed with a taper portion 11a whose outer diameter is reduced toward the distal end thereof. A rear end portion of the second probe-constituting unit 12 is formed with a rear end taper portion 12a whose outer diameter is reduced toward a rear end thereof.

As shown in FIG. 4, the weight member 13 includes a plurality of (three in this embodiment) replacement members 14A to 14C. The three replacement members 14A to 14C are set to be different state in weight from one another, for example, by changing at least one of the longitudinal dimensions L thereof in an axial central direction of the probe main body 9 and dimensions D in a diametrical direction thereof.

FIG. 4 is a diagram showing one example of three replacement members 14A to 14C of the weight member 13. A first replacement member 14A is set such that the longitudinal dimension in the axial central direction of the probe main body 9 is L1 and the dimension in the diametrical direction is D1. A second replacement member 14B is set such that the longitudinal dimension in the axial central direction of the probe main body 9 is L2 and the dimension in the diametrical direction is D2. A third replacement member 14C is set such that the longitudinal dimension in the axial central direction of the probe main body 9 is L3 and the dimension in the diametrical direction is D3. Here, the dimensions L of the three replacement members 14A to 14C in the axial central direction of the probe main body 9 are set to satisfy a relationship of L1<L2<L3. In addition, the dimensions D of the three replacement members 14A to 14C in the diametrical direction are set to satisfy a relationship of D1<D2<D3.

In the adjusting portion 10, one of three replacement members 14A to 14C of the weight member 13 are selectively used according to variations due to working tolerance of the probe main body 9 after worked so that the weight and the dimension of the probe main body 9 are changed to adjust a resonance frequency of the probe main body 9.

As shown in FIG. 3, the replacement members 14A to 14C have a replacement member main unit 15 cylindrically formed, respectively. A cylindrical front end coupling ring 15a is formed at a front end portion of the replacement member main unit 15. A cylindrical rear end coupling ring 15b is similarly formed at a rear end portion of the replacement member main unit 15. An inner diameter of the front end coupling ring 15a is formed to be approximately equal to an outer diameter of an end portion of a rear end taper portion 12a of the second probe-constituting unit 12. Similarly, an inner diameter of the rear end coupling ring 15b is formed to be approximately equal to an outer diameter of an end portion of a front end taper portion 11a of the first probe-constituting unit 11.

The rear end coupling ring 15b of the replacement member main unit 15 is fitted with the first probe-constituting unit 11 in a state that the front end taper portion 11a of the first probe-constituting unit 11 has been inserted into the rear end coupling ring 15b, and the replacement member main unit 15 and the first probe-constituting unit 11 are connected by welding the fitted portion of both the units. Similarly, the front end coupling ring 15a of the replacement member main unit 15 is fitted with the second probe-constituting unit 12 in a state that the rear end taper portion 12a of the second probe-constituting unit 12 has been inserted into the front end coupling ring 15a, and the replacement member main unit 15 and the second probe-constituting unit 12 are connected by welding the fitted portion of the units. Thereby, the first and second probe-constituting units 11 and 12 are connected to each other via the weight member 13. Incidentally, as a method for connecting the first and second probe-constituting units 11 and 12 and weight member 13, screw joining and pressure-fitting can be adopted.

Next, an operation of the ultrasonic treatment tool 1 according to the present embodiment with the above mentioned configuration will be explained. In the ultrasonic treatment tool 1 according to the present embodiment, one of three replacement members 14A to 14C of the weight member 13 is selectively used according to variations due to working tolerance of the probe main body 9 after worked. The selected replacement member 14A (or the replacement member 14B or 14C) is interposed between the first and second probe-constituting units 11 and 12 of the probe main body 9. Thereby, the weight and dimension of the probe main body 9 can be changed to adjust the resonance frequency of the probe main body 9.

The following effects can be achieved with the above-mentioned configuration. That is, in the ultrasonic treatment tool 1 of the ultrasonic operating apparatus according to the present embodiment, it is possible to change or adjust the resonance frequency of the probe main body 9 after working the probe main body 9. Therefore, even if variations among respective products due to working tolerance occur at a working time of the probe main body 9, one of three replacement members 14A to 14C of the weight member 13 is selectively used according to the variations due to the working tolerance after working, so that the resonance frequency of the probe main body 9 including the variations can be adjusted to a resonance frequency of the probe main body 9 put in a reference state. The dimension of the probe main body 9 can also be adjusted. As a result, yield at a manufacturing time of probe main bodies 9 can be improved. The three replacement members 14A to 14C can allow setting of a relatively large equivalent mass to the whole vibration system. Therefore, an adjustment range of the resonance frequency of the probe can be set to be relatively large.

Incidentally, the numbers of probe-constituting units and weight members 13 for the probe main body 9 should not be limited and the units and members may be connected by a required number according to the length of the operating tool 1.

FIG. 5 is a view showing a first modification of the probe main body 9 of the ultrasonic operating apparatus according to the first embodiment. In the present modification, a joining member 21 is interposed between the probe main body 9 and the ultrasonic transducer 2. A length L11 of the joining member 21 is set to a length of an integral multiple of about a half wavelength of ultrasonic vibrations of the ultrasonic transducer 2. Thereby, an occupation ratio of an equivalent mass of the joining member 21 to that of the vibration system becomes large. Therefore, the joining member 21 can achieve adjustment to a desired resonance frequency singly.

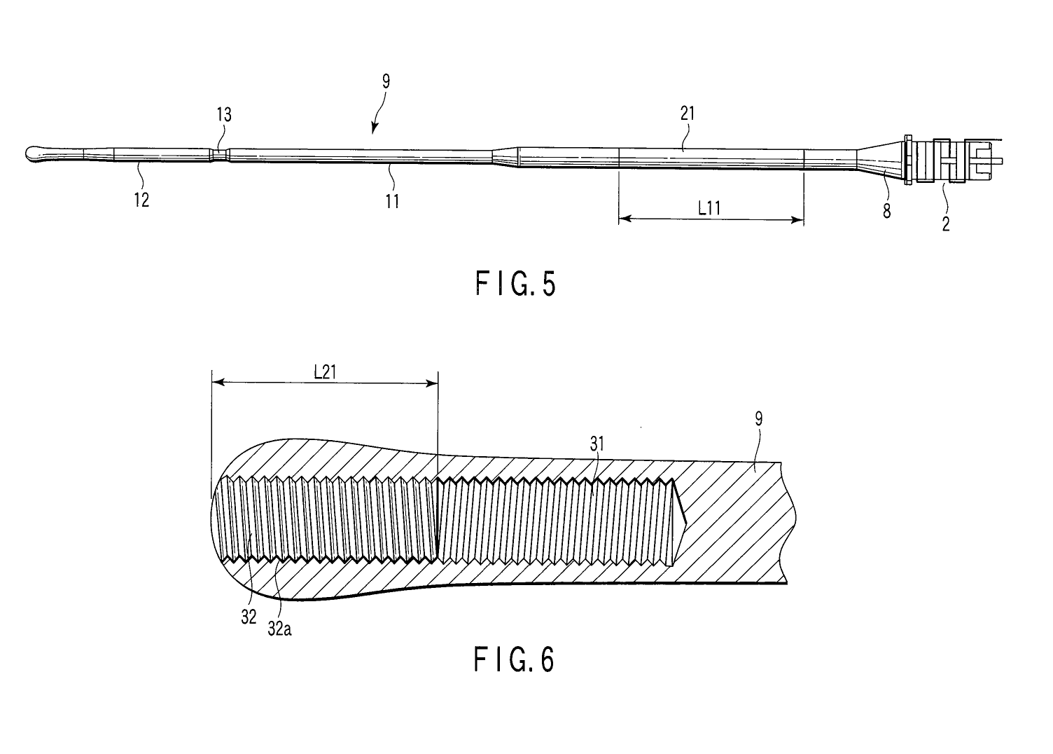

FIG. 6 is a sectional view showing a second modification of the probe main body 9 of the ultrasonic apparatus according to the first embodiment. In the present second modification, a screw hole portion (an adjusting hole for resonance frequency) 31 is provided at the distal end portion of the probe main body 9. The screw hole portion 31 is provided at an axial center portion at the distal end portion of the probe main body 9 so as to extend along an axial central direction thereof.

A coupling member 32 with a high density such as stainless steel (SUS) is coupled in the screw hole portion 31. A male screw portion 32a is formed on the outer peripheral face of the coupling member 32. The coupling member 32 is coupled to the probe main body 9 by driving the male screw portion 32a into the screw hole portion 31. In the present modification, the weight of the coupling member 32 is adjusted by changing the whole length L21 of the coupling member 32.

In the first embodiment, as shown in FIG. 4, the configuration that three replacement members with different weights that are different in the longitudinal dimension L in the axial central direction of the probe main body 9 and the dimension D in the diametrical direction thereof has been shown, for example. Alternatively, such a configuration that the weight member 13 has a plurality of replacement members 14 having the same dimension L in the axial central direction of the probe main body 9 and the dimension D in the diametrical direction thereof may be adopted. In this case, the adjusting portion 10 uses the selected number of replacement members 14 to change the length and the weight of the probe main body 9, thereby adjusting the resonance frequency.

In the present modifications, even when variations among respective products due to working tolerance occur at a working time of the probe main body 9, replacement members 14 of the weight member 13 of the number selected according to variations due to working tolerance after working are used so that the resonance frequency of the probe main body 9 including the variations can be changed and adjusted in the same manner as the first embodiment. The dimension of the probe main body 9 can also be adjusted. As a result, yield at manufacturing time of the probe main bodies 9 can be improved.

Such a configuration that the weight member 13 has a plurality of replacement members 14 formed from different metal materials, respectively, and the adjusting portion 10 selectively uses one of the replacement members 14 to adjust the resonance frequency of the probe main body 9.

### Second Embodiment

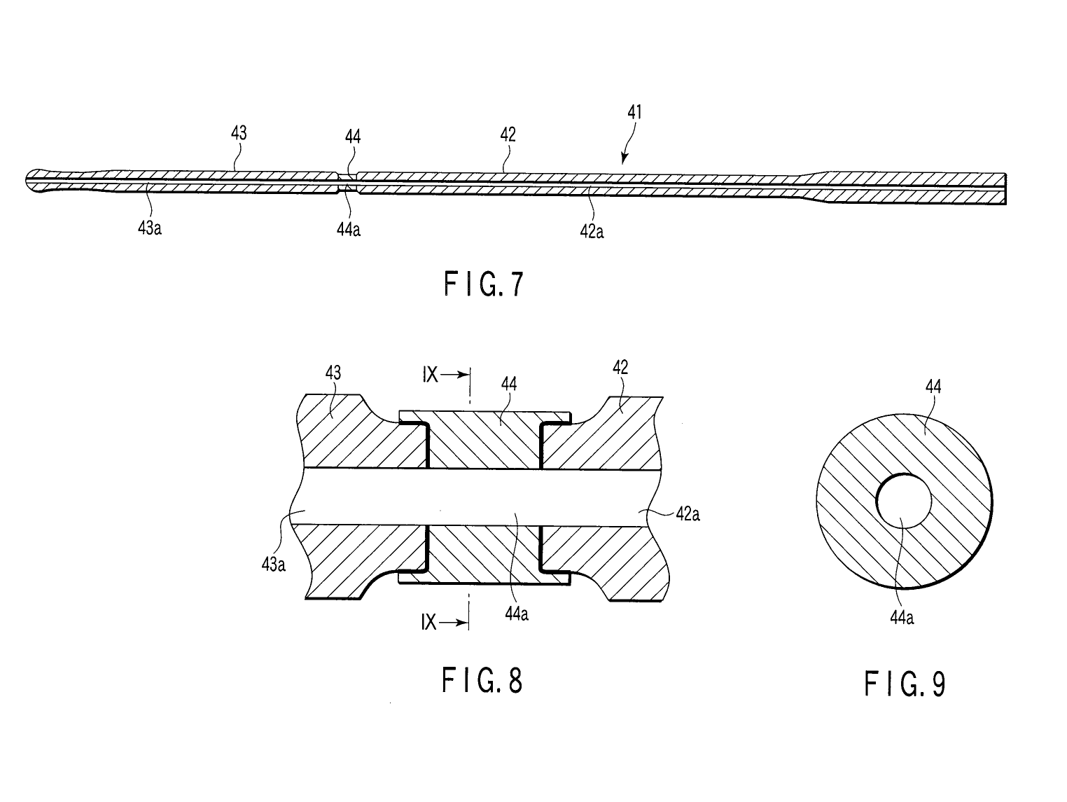

FIG. 7 to FIG. 9 are views showing a second embodiment of the present invention. In the present embodiment, a hollow probe main body 41 is provided. As shown in FIG. 7, a probe main body 41 has a first probe-constituting unit 42, a second probe-constituting unit 43, and a weight member (adjusting member) 44 interposed between the first probe-constituting unit 42 and the second probe-constituting unit 43. A hollow conduit 42a is formed at an axial central portion of the first probe-constituting unit 42, a hollow conduit 43a is formed at an axial center portion of the second probe-constituting unit 43, and a hollow conduit 44a (see FIG. 9) is formed at an axial center portion of the weight member 44. The first and second probe-constituting units 42 and 43 are connected via the weight member 44 as in the first embodiment.

The weight member 44 has a plurality of replacement members with different weights that are different in a longitudinal dimension L in a lengthwise direction along an axial central direction of the probe main body 41 and a dimension D in a diametrical direction thereof, for example, (see FIG. 4).

As shown in FIG. 8, the hollow conduit 42a of the first probe-constituting unit 42, the hollow conduit 44a of the weight member 44, and the hollow conduit 43a of the second probe-constituting unit 43 are caused to communicate with one another. Thereby, the conduit of the probe main body 41 is put in communication over the whole length of the probe from a proximal end to a distal end thereof.

In the present embodiment, even if variations among respective products due to working tolerance occur at a working time of the probe main body 41, either of the plurality of replacement members of the weight member 44 is selectively used according to the variations due to the working tolerance after working, so that the resonance frequency of the probe main body 41 including the variations can be changed and adjusted. Thereby, the worked probe main body 41 can be adjusted to have the same resonance frequency as that of the probe main body 41 put in a reference state, so that yield at a manufacturing time of the probe main bodies 41 can be improved as in the first embodiment.

### Third Embodiment

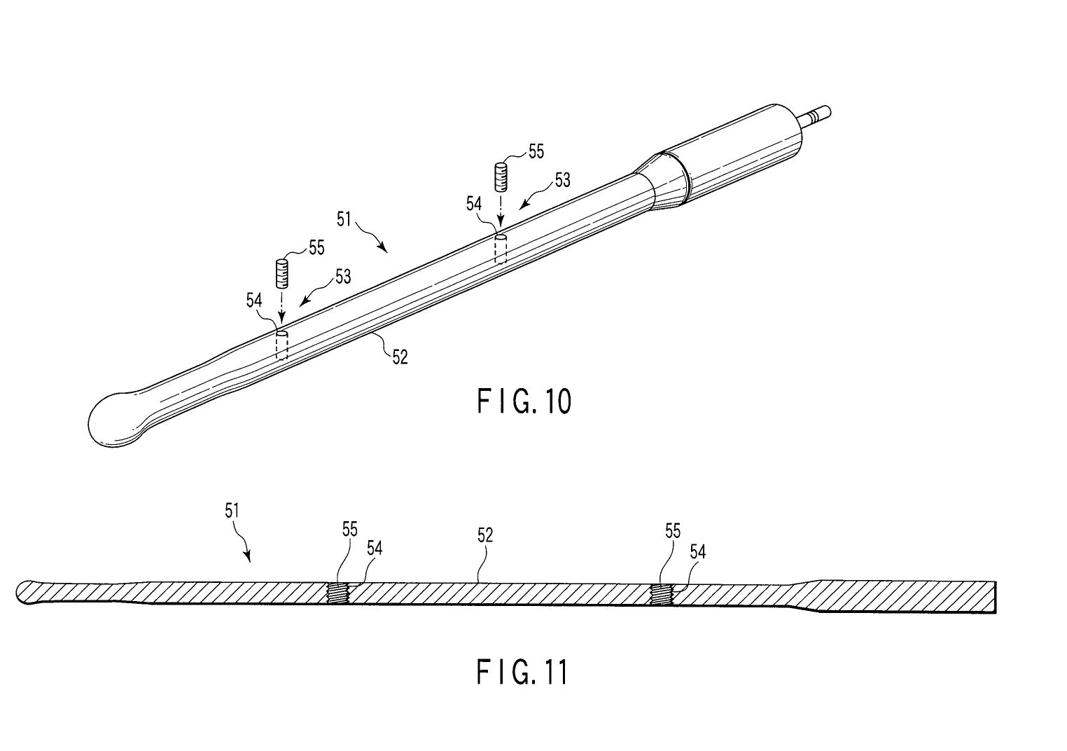

FIG. 10 and FIG. 11 are views showing a third embodiment of the present invention. In an ultrasonic treatment tool according to the third embodiment, a configuration of a probe 51 is different from that of the probe 3 in the first embodiment. A whole configuration of an ultrasonic treatment tool according to this embodiment except for a changed portion of the probe 51 is similar to that of the ultrasonic treatment tool 1 according to the first embodiment.

As shown in FIG. 10, in the probe 51 according to the present embodiment has adjusting portions 53 for a resonance frequency at a plurality of positions (two positions in the present embodiment) on a rod-shaped probe main body 52 that is an ultrasonic wave transmitting member with a length of an integral multiple of about a half wavelength of ultrasonic vibrations of the ultrasonic transducer 2. The adjusting portion 53 has an adjusting hole 54 formed in the probe main body 52 and a joining member 55 that can be joined to the adjusting hole 54.

The probe main body 52 is made from, for example, metal material such as titanium (Ti) alloy, duralumin, or stainless steel (SUS). The adjusting hole 54 is disposed on the probe main body 52 at an antinode position of vibrations when ultrasonic vibrations are transmitted to the probe main body 52. The adjusting hole 54 is formed as a screw hole. Incidentally, the adjusting hole 54 may be disposed at a position on the probe main body 52 except for the antinode position of ultrasonic vibrations thereof.

The joining member 55 is formed as a male screw driven into a screw hole of the adjusting hole 54. The male screw of the joining member 55 is driven into the screw hole of the adjusting hole 54 so that the joining member 55 is joined to the adjusting hole 54 in a screw-joined state. The joining member 55 is made from metal material having the same density as that of the material for the probe main body 52 or metal material having density different from that of the material for the probe main body 52. The joining member 55 may be made from resin.

In the present embodiment, the configuration that the joining member 55 has the screw-joining portion at the coupling portion with the adjusting hole 54 has been shown, but the present invention is not limited to the configuration and a configuration that the coupling portion of the joining member 55 and the adjusting hole 54 has a connecting portion comprising one of welding and fitting, the fitting being means for pressure-fitting, into a small diameter hole, a shaft body with a diameter larger than the diameter of the small diameter hole may be adopted. The adjusting portion 53 adjusts the resonance frequency by coupling the joining member 55 into the adjusting hole 54 to change the weight of the probe main body 52. The number and the positions of the adjusting holes 54 are not limited, and the adjusting holes are provided by a required number thereof.

Next, an operation of the present embodiment with the above-mentioned configuration will be explained. In the ultrasonic treatment tool 1 according to the present embodiment, the resonance frequency of the probe main body 52 can be changed by selecting the joining member 55 to be assembled to the adjusting hole 54 according to variations due to working tolerance of the probe main body 52 after worked. The selected joining member 55 is screwed into the adjusting hole 54 of the probe main body 52 to be joined to the probe main body 52. Thereby, the resonance frequency of the probe main body 52 can be adjusted by changing the weight of the probe main body 52.

With the above-mentioned configuration, the following effects can be obtained. That is, in the ultrasonic treatment tool of the ultrasonic operating apparatus according to the present embodiment, change or adjustment of a resonance frequency can be made possible without changing the dimension of the probe main body 52 after the probe main body 52 has been worked. Therefore, even if variations among respective products occur due to working tolerance at a working time of the probe main bodies 52, the coupling member 55 is selectively used according to the variations due to the working tolerance after working, so that the resonance frequency of the probe main body 52 including the variations can be adjusted to a resonance frequency of the probe main body 52 put in a reference state. As a result, yield at a manufacturing time of probe main bodies 52 can be improved. The joining member 55 can allow setting of a relatively large equivalent mass to the whole vibration system. Therefore, an adjustment range of the resonance frequency of the probe can be set to be relatively large.

### Fourth Embodiment

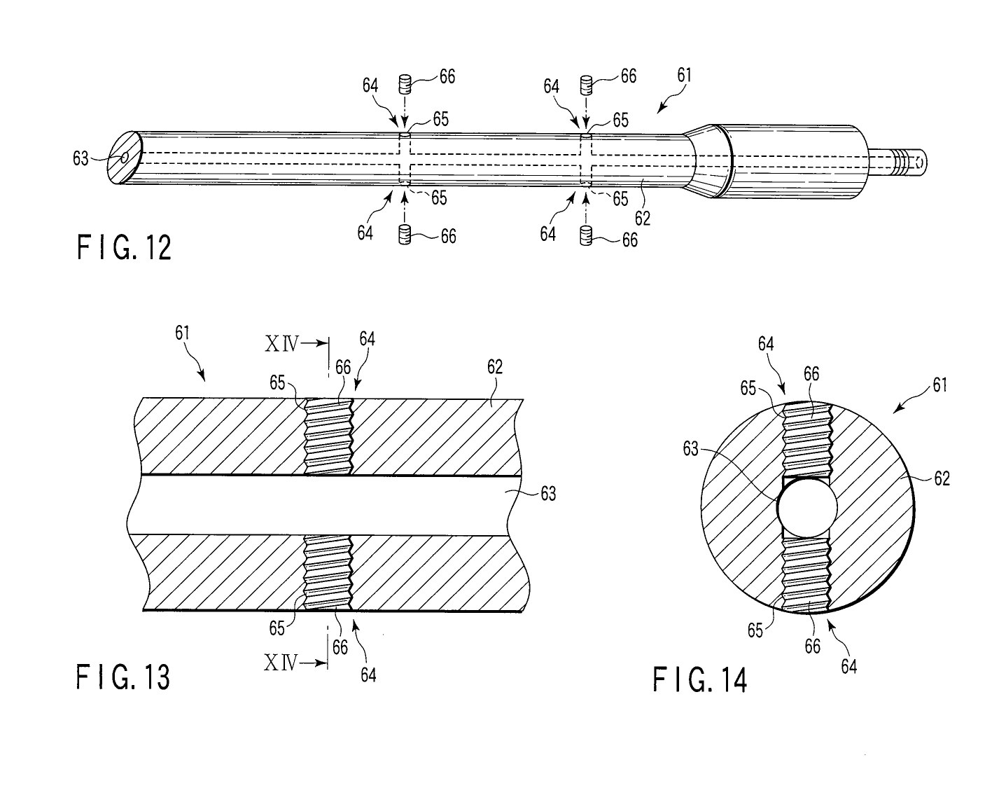

FIG. 12 to FIG. 14 are views showing a fourth embodiment of the present invention. The present embodiment has a configuration obtained by changing the configuration of the probe 51 according to the third embodiment (see FIG. 10 and FIG. 11) in the following manner. Incidentally, the fourth embodiment has the same configuration as that of the third embodiment except for a changed portion of the former. Here, only a configuration of a portion different from the probe 51 according to the third embodiment will be explained.

That is, a probe main body 62 of a probe 61 according to the fourth embodiment is formed of a hollow member formed at an axial center portion thereof with a conduit 63. Adjusting portions 64 for a resonance frequency are provided at a plurality of (four in the embodiment) portions on an outer peripheral face of the probe main body 62. The adjusting portion 64 has an adjusting hole 65 formed on the probe main body 62 and a joining member 66 that can be joined in the adjusting hole 65. An inner end portion of each adjusting hole 65 communicates with the hollow conduit 63 of the probe main body 62.

The probe main body 62 is made from metal material such as, for example, titanium (Ti) alloy, duralumin, and stainless steel (SUS). The adjusting hole 65 is disposed at an antinode position of vibrations when ultrasonic vibrations are transmitted to the probe main body 62. The adjusting hole 65 is formed as a screw hole. Incidentally, the adjusting hole 65 may be disposed at a position on the probe main body 62 except for the antinode position of ultrasonic vibrations thereof.

The joining member 66 is formed as a male screw driven into a screw hole of the adjusting hole 65. The male screw of the joining member 66 is driven into the screw hole of the adjusting hole 65 so that the joining member 66 is joined to the adjusting hole 65 in a screw-joined state. The joining member 66 is made from metal material having the same density as that of the material for the probe main body 62 or metal material having density different from that of the material for the probe main body 62. The joining member 66 may be made from resin.

Next, an operation of the embodiment with the above-mentioned configuration will be explained. In the ultrasonic treatment tool 1 according to the embodiment, the resonance frequency of the probe main body 62 can be changed by selecting a joining member 66 to be assembled in the adjusting hole 65 according to variations due to working tolerance after the probe main body 62 has been worked. The selected joining member 66 is screwed into the adjusting hole 65 of the probe main body 62 to be joined thereto. Thereby, the resonance frequency of the probe main body 62 can be adjusted by changing the weight of the probe main body 62.

Incidentally, such a state that the outside of the probe main body 62 and the hollow conduit 63 remain in communication with each other via the adjusting holes 65 can be prevented by joining the joining members 66 into all the adjusting holes 65.

With the above-mentioned configuration, the following effects can be obtained. That is, in the ultrasonic treatment tool of the ultrasonic operating apparatus according to the embodiment, change or adjustment of a resonance frequency can be made possible without changing the dimension of the probe main body 62 after the probe main body 62 has been worked. Therefore, even if variations among respective products occur due to working tolerance at a working time of the probe main bodies 62, the joining member 66 is selectively used corresponding to the variations due to the working tolerance after working, so that the resonance frequency of the probe main body 62 including the variations can be adjusted to a resonance frequency of the probe main body 62 put in a reference state. As a result, yield at a manufacturing time of probe main bodies 62 can be improved. The joining member 66 can allow setting of a relatively large equivalent mass to the whole vibration system. Therefore, an adjustment range of the resonance frequency of the probe 61 can be set to be relatively large.

Incidentally, the present invention is not limited to the embodiments, and it can be variously modified and implemented without departing from the gist of the present invention.

Next, other characteristic technical items of the present application will be described below.

### Note

(Additional Item 1) An ultrasonic treatment tool comprising a probe where a first ultrasonic transmitting member and a second ultrasonic transmitting member, each having a length of integral multiple about a half wavelength of ultrasonic vibrations, are connected to each other via a weight member.

(Additional Item 2) The ultrasonic treatment tool comprising according to the addition item 1, wherein the first and second ultrasonic transmitting members and the weight member have hollow conduit, respectively, and the conduits of the ultrasonic transmitting member and the conduit of the weight member communicate with one another.

(Additional Item 3) An ultrasonic treatment tool having a probe comprising an ultrasonic wave transmitting member having a hole and an adjusting member that can be fitted into the hole.

(Additional Item 4) The ultrasonic treatment tool according to the additional item 3, wherein the ultrasonic wave transmitting member is hollow.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic operating apparatus comprising:
an ultrasonic transducer that generates ultrasonic vibrations; and
a probe that has a distal end portion and a proximal end portion, the proximal end portion being coupled to the ultrasonic transducer, and to which ultrasonic vibrations output from the ultrasonic transducer are transmitted, wherein
the probe has a probe main body having a length of an integral multiple of about a half wavelength of the ultrasonic vibrations and
an adjusting portion that changes a weight and a dimension of the probe main body to adjust a resonance frequency of the probe main body.

2. The ultrasonic operating apparatus according to claim 1,
the probe main body comprises a first probe-constituting unit, a second probe-constituting unit, and an adjusting member that is interposed between the first probe-constituting unit and the second probe-constituting unit and adjusts a resonance frequency of the probe main body, the adjusting member being disposed at an antinode position of the ultrasonic vibrations, and
the adjusting portion changes the adjusting member to adjust a resonance frequency of the probe main body.

3. The ultrasonic operating apparatus according to claim 2, wherein
the adjusting member has a weight member interposed between the first probe-constituting unit and the second probe-constituting unit.

4. The ultrasonic operating apparatus according to claim 3, wherein
the weight member includes a coupling portion comprising one of at least welding, screw joining, and fitting at a coupled section of the first probe-constituting unit and the second probe-constituting unit, the fitting being means for pressure-fitting, into a small hole, a shaft body with a diameter larger than that of the small diameter hole.

5. The ultrasonic operating apparatus according to claim 3, wherein
in the probe main body, the first and second probe-constituting units are made from metal material, and
the weight member is made from the same metal material as material used for the probe-constituting units.

6. The ultrasonic operating apparatus according to claim 5, wherein
the metal material is one of at least titanium alloy, duralumin, and stainless steel.

7. The ultrasonic operating apparatus according to claim 3, wherein
in the probe main body, the first and second probe-constituting units are made from metal material, and
the weight member is made from metal material different from the material used for the probe-constituting units.

8. The ultrasonic operating apparatus according to claim 3, wherein
the weight member has a plurality of replacement members with different weights that are different in at least one of a dimension in a longitudinal direction along an axial central direction of the probe main body and a dimension in a diametrical direction thereof, and
the adjusting portion selects and uses one of the replacement members to adjust a resonance frequency of the probe main body.

9. The ultrasonic operating apparatus according to claim 3, wherein
the weight member has a plurality of replacement members equal in dimension in a longitudinal direction along an axial central direction of the probe main body and dimension in a diametrical direction thereof, and
the adjusting portion selects the number of replacement members and uses the selected replacement member to change a length and a weight of the probe main body, thereby adjusting the resonance frequency.

10. The ultrasonic operating apparatus according to claim 3, wherein
the weight member has a plurality of replacement members made from different metal materials, and
the adjusting portion selects and uses one of the replacement members to adjust the resonance frequency of the probe main body.

11. The ultrasonic operating apparatus according to claim 2, wherein
the probe main body has hollow conduits provided in the first probe-constituting unit, the second probe-constituting unit, and the adjusting member, respectively, and
the hollow conduit of the first probe-constituting unit, the hollow conduit of the second probe-constituting unit, and the hollow conduit of the adjusting member are in communication with one another, respectively.

12. The ultrasonic operating apparatus according to claim 1, wherein
the probe main body has an adjusting hole for a resonance frequency, the adjusting hole being disposed at an antinode position of the ultrasonic vibrations, and
the adjusting portion has a joining member that can be joined to the adjusting hole, the joining member adjusting changing a weight of the probe main body to adjust the resonance frequency.

13. The ultrasonic operating apparatus according to claim 12, wherein
the joining member includes a coupling portion comprising one of at least welding, screw joining, and fitting at a coupled section with the adjusting hole, the fitting being means for pressure-fitting, into a small hole, a shaft body with a diameter larger than that of the small diameter hole.

14. The ultrasonic operating apparatus according to claim 12, wherein
the probe main body has a hollow conduit communicating with the adjusting hole, and
the joining member is coupled to the adjusting hole to close the adjusting hole.

15. The ultrasonic operating apparatus according to claim 14, wherein
the joining member includes a coupling portion comprising one of at least welding, screw joining, and fitting at a coupled portion with the adjusting hole, the fitting being means for pressure-fitting, into a small hole, a shaft body with a diameter larger than that of the small diameter hole.
